(19) United States
(12) Patent Application Publication
(10) Pub. No.: US 2026/0069423 A1
(43) Pub. Date: Mar. 12, 2026

(54) ANTIOXIDANT STABILIZED SPINAL IMPLANT COMPONENTS

(71) Applicant: 3Spine, Inc., Chattanooga, TN (US)

(72) Inventors: Christian Davis, Cambridge, MA (US); Marc M. Peterman, Duxbury, MA (US); Steven C. Humphreys, Soldotna, AK (US); Scott Hodges, Chattanooga, TN (US); Ron Yarbrough, Round Rock, TX (US)

(21) Appl. No.: 19/321,100

(22) Filed: Sep. 5, 2025

Related U.S. Application Data
(63) Continuation of application No. PCT/US24/18902, filed on Mar. 7, 2024.
(60) Provisional application No. 63/450,608, filed on Mar. 7, 2023.

(51) Int. Cl. A61F 2/44; A61L 27/18; A61L 27/54

(52) U.S. Cl. CPC A61F 2/4425; A61L 27/18; A61L 27/54

(57) ABSTRACT

Disclosed are apparatus, systems and methods for crosslinked, oxidatively stable, spinal implants. More specifically, the invention provides spinal implants, systems and methods of forming spinal implants from a polymer blend stabilized with Vitamin E to improve performance, wear and/or fatigue resistance.

### CROSS-REFERENCE TO RELATED APPLICATIONS

This application is a continuation of Patent Cooperation Treaty Patent Application No. PCT/US24/18902 entitled “ANTIOXIDANT STABILIZED SPINAL IMPLANT COMPONENTS” filed Mar. 7, 2024, which claims the benefit of U.S. Provisional Patent Application Ser. No. 63/450,608 entitled “Antioxidant Stabilized Spinal Implant Methods” filed Mar. 7, 2023, the disclosures of which are each incorporated by reference herein in their entireties.

### TECHNICAL FIELD

The invention relates to methods, devices, and systems for an antioxidant stabilized spinal implant and/or components thereof. More specifically, the invention relates antioxidant stabilized spinal implants and the methods to form antioxidant stabilized spinal implants to decrease wear, increase implant longevity and decrease inflammatory reactions.

### BACKGROUND OF THE INVENTION

Many prosthetic joint replacements, e.g., total hip prosthetics or total knees, currently implanted in patients include a highly polished metal or ceramic component articulating on an ultra-high molecular weight polyethylene (UHMWPE) material or blend. Wear and abrasion resistance, coefficient of friction, impact strength, toughness, density, biocompatibility and biostability are some of the properties that make UHMWPE a suitable material for such implants. Although UHMWPE has been used in implants for many years, there is continuing interest in the wear and durability characteristics of implants incorporating UHMWPE.

One technique employed to improve the durability and other physical characteristics of UHMWPE implants has been to expose such implants to radiation, for example gamma radiation or electron beam radiation, to induce crosslinking in the UHMWPE that creates bonds or a short sequence of bonds to link one polymer chain to another. In many cases, similar radiation sources can also be used to sterilize UHMWPE implants prior to distribution and/or use. However, the irradiation process may lead to increased rates of oxidation in the UHMWPE implant and generate free radicals, which react in the presence of oxygen to form peroxyl radicals. These free radicals and peroxyl radicals may react with the polyethylene backbone and with each other to form oxidative degradation products and additional radical species. This cycle of oxidation product and radical species formation may occur over several years (both prior to and after implantation) as oxidation levels in the implant increase. In the long-term, these free radicals presumably migrate to the crystalline/amorphous interface of the implant and cause embrittlement and oxidative degradation by reacting with diffused oxygen.

### BRIEF DESCRIPTION OF THE SEVERAL VIEWS OF THE DRAWINGS

FIGS. 1A-1B illustrates various flowcharts with different embodiments of a method of forming an antioxidant stabilized spinal implant.

FIGS. 2A-2I depicts various plan views of one embodiment of a superior base element;

FIGS. 3A-3E depicts various plan views of one embodiment of a preformed spinal implant or a portion of a preformed spinal implant;

FIGS. 3F-3H depicts cross-section views of the preformed spinal implant or a portion of a preformed spinal implant of FIGS. 3A-3E;

FIGS. 4A-4F depicts various plan views of one embodiment of a final spinal implant or a portion of a final implant;

FIG. 5 depicts a side view of the crosslink profile of a final spinal implant or a portion of a final implant;

FIGS. 6A-6B depicts different plant views of one embodiment of a final implant assembly or final implant; and

FIG. 7 depicts a side view of an alternative crosslink profile of a final spinal implant or a portion of a final implant.

### DETAILED DESCRIPTION OF THE INVENTION

Various embodiments herein include the realization of a need in the art to further optimize a spinal implant to reduce oxidation degradation, as well as improve mechanical properties by the addition of a stabilizing component to the spinal implant material prior to irradiation. In various embodiments, the incorporation of a polymer insert into a spinal implant device can significantly improve the performance and/or durability of the implant, as well as potentially allow for controlled deformation or “flexing” of various implant components during use. In various embodiments, an implant manufactured using various of the methods described herein can balance wear resistance, oxidation resistance and/or other desired mechanical properties to ensure that the implant accomplished it desired purpose. In some embodiments, a crosslink density on the surface and/or throughout the preformed or finished/final implant (or portions thereof) is controlled, desirably resulting in the improvement of fatigue strength and/or wear resistance.

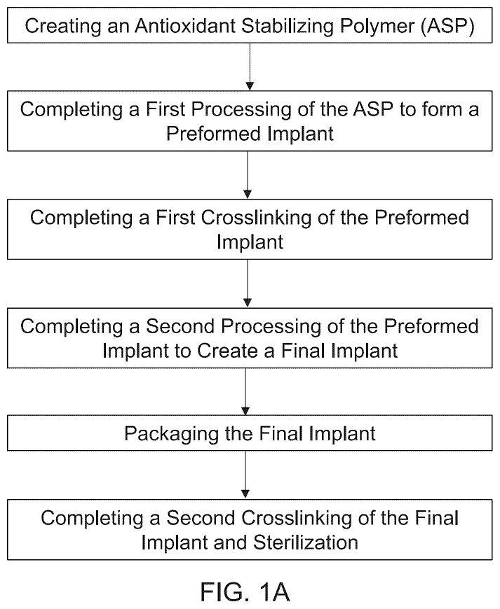

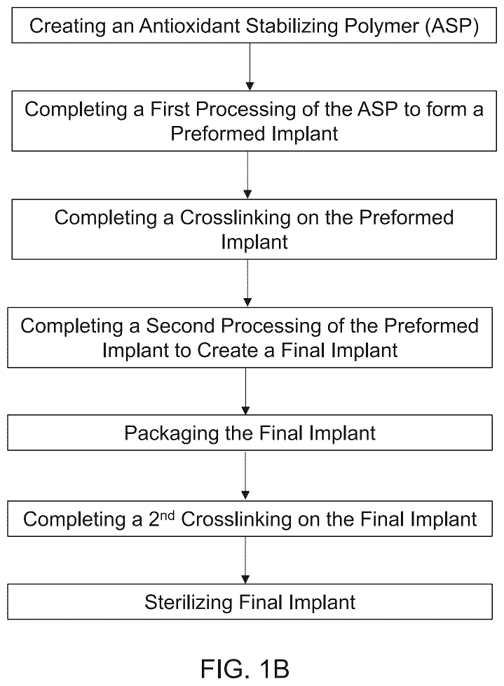

With reference to FIG. 1A, one exemplary method of forming a cross-linked stabilized spinal implant comprises the steps of: creating an antioxidant stabilizing polymer (ASP); completing a first processing with the ASP to create a portion of a preformed implant; completing a cross-linking on the preformed implant with a selected technique; completing a second processing of the preformed implant to create a final implant; packaging a portion of a final implant or the final implant; and sterilizing the portion of a final implant or the final implant with a selected technique.

With reference to FIG. 1B, a method of forming a cross-linked stabilized spinal implant can include one or more of the steps of: creating an antioxidant stabilizing polymer (ASP); completing a first processing with the ASP to create a portion of a preformed implant; completing a first cross-linking on the preformed implant with a selected technique; completing a second processing of the preformed implant to create a final implant; packaging a portion of a final implant or the final implant; completing a second crosslinking of the portion of a final implant or the final implant with a selected technique; and sterilizing the final implant.

In one embodiment, an exemplary method of forming a cross-linked stabilized spinal implant may include creating an antioxidant stabilizing polymer (ASP). The antioxidant stabilized polymer (ASP) may comprise a polymer and at least one stabilizing component. The at least one stabilizing component may include an antioxidant. The antioxidant may comprise Vitamin E (a-tocopherals, a-tocopheral derivatives, or tocotrienols). The antioxidant stabilized polymer (ASP) may further comprise at least one additive and/or solvent. The creating of an ASP may comprise a blending, doping or diffusing process.

The polymers may comprise a material, the material comprises a thermoset, a thermoplastic or an elastomer. The polymer material may comprise a polyolefin, polybutadiene (PBD), nylon, polyethylene (PE), low-density polyethylene, high-density polyethylene, ultrahigh molecular weight polyethylene (UHMWPE), polypropylene (PP), polyurethanes (PU), polyester, polyvinylchloride (PVC), polyamide, polyether ketone and/or any combination thereof. The polymer material may further comprise a resin, a powder, pellets, flakes, particles, and/or any combination thereof. In one embodiment, the polymer material comprises UHMWPE. UHMWPE is the preferred material for bearing surfaces of joint implants due to its high toughness, superior abrasion resistance and superior fatigue crack propagation.

The at least one stabilizing component may comprise an antioxidant. The antioxidant may comprise a natural antioxidant or a synthetic antioxidant. Natural antioxidants may include ascorbic acid (Vitamin C), Vitamin E (tocopherols, tocopheral derivatives or tocotrienols). Synthetic antioxidants include propyl gallate (PG), tertiary butylhydroquinone (TBHQ), butylated hydroxyanisole (BHA), butylated hydroxytoluene (BHT), α-lipoic acid, N-acetyl cysteine, melatonin, gallic acid, captopril, taurine, catechin, quercetin, and/or any combination thereof. In one exemplary embodiment, the stabilizing component comprises Vitamin E.

The stabilizing component may comprise a concentration (% Wt) of an effective stabilizing amount and/or a percent dispersion (ppm or parts per million). The concentration/dispersion may comprise a range of 0.001 to 5% Wt and/or 10 ppm to 50,000 ppm; the concentration/dispersion may comprise a range of 0.001 to 3% Wt and/or 10 ppm to 30,000 ppm; the concentration/dispersion may comprise a range of 0.001 to 1% Wt and/or 10 ppm to 10,000 ppm; the concentration/dispersion may comprise a range of 0.001 to 0.5% Wt and/or 10 ppm to 5,000 ppm; the concentration/dispersion may comprise a range of 0.01 to 0.3% Wt and/or 100 ppm to 3,000 ppm; the concentration/dispersion may comprise a range of 0.1 to 0.3% Wt and/or 1000 ppm to 3,000 ppm. Alternatively, the concentration/dispersion may comprise at least 0.001% Wt (and/or 10 ppm) or greater; the concentration/dispersion may comprise at least 0.01% Wt (and/or 100 ppm) or greater; the concentration/dispersion may comprise at least 0.1% Wt (and/or 1000 ppm) or greater; the concentration/dispersion may comprise at least 0.2% Wt (and/or 2000 ppm) or greater. In at least one preferred embodiment, the stabilizing component may comprise a concentration/dispersion of at least 900 ppm, with a preferred concentration/dispersion range of 900 ppm to 1100 ppm or greater.

In one embodiment, the creating of an ASP may include a blending process, which can included blending processes known by those of ordinary skill in the art. The antioxidant may be prepared in a solution, the solution may include regents, solvents, and/or an additive. The antioxidant solution and the polymer material can be mixed or “blended” together while undergoing one or more preparation steps of drying, pressurizing, heating, cooling in a controlled environment.

In another embodiment, the creating of an ASP may include a diffusion process, including diffusion processes known by those of ordinary skill in the art. Diffusion may include infusion, soaking and/or doping. The polymer material may be soaked in the desired antioxidant with a subsequent homogenization step using a controlled environment that is below the melting point of the polymer. The crosslinking efficiency of the polymer is desirably not adversely affected in this method since the stabilizing component may not be present during irradiation. Therefore, the amount of the stabilizing component that can be incorporated into the material is not limited by concerns for crosslink density. Furthermore, a homogenization step may be required after incorporation to obtain adequate stabilizing component concentration throughout the final implants, preformed articulation components and/or final articulation components.

In another embodiment, the method of forming a cross-linked stabilized spinal implant may comprise a step of: completing a first processing with the antioxidant stabilized polymer (ASP) to form a preformed implant. The step of completing the first processing may comprise the steps of: obtaining a first element or base element and the ASP; completing a molding process to obtain the preformed implant. The first processing may further comprise completing an insert molding or over-molding process. The preformed implant comprises a base element and a preformed or molded ASP. The preformed implant may comprise the molded or preformed ASP.

In one embodiment, the preformed implant may include a base element. The base element may comprise a metal element, a polymer element, a ceramic element and/or any combination thereof. The base element may comprise a finished component, a semi-finished component or a finished component.

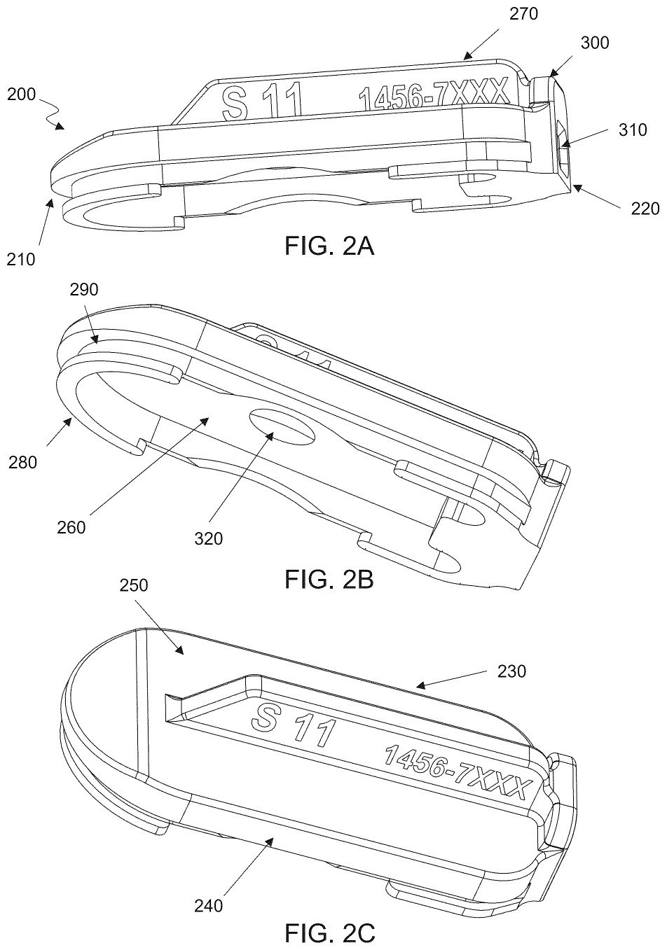

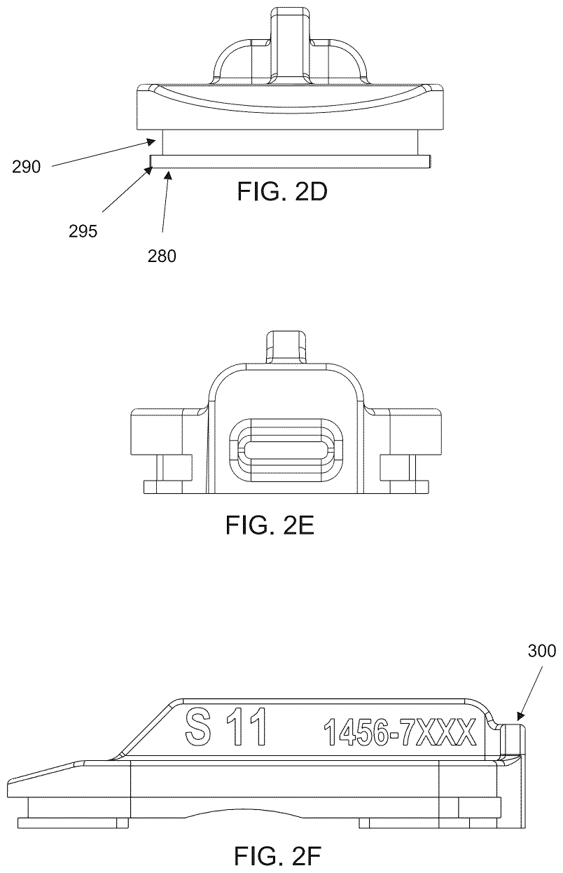

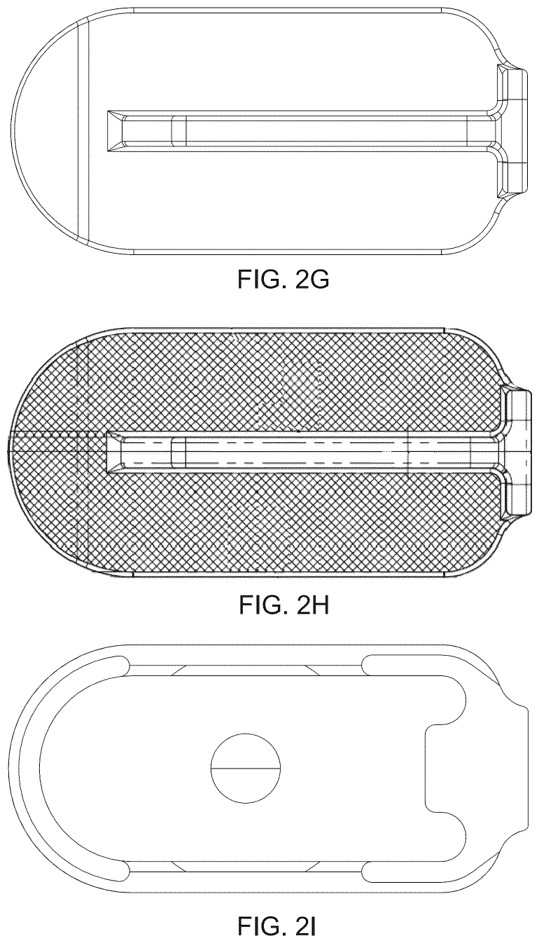

In one exemplary embodiment, the base element comprises a metallic base element, the metallic base element including a superior component or superior base 200 as shown in FIGS. 2A-2I. The superior base 200 can comprise a first end or anterior end 210, a second end or posterior end 220, a third end or medial end/side 230 and/or a fourth end or lateral end/side 240. The superior base may further comprise a top surface 250 and a bottom surface 260. At least a portion of the top surface may be flat or planar. At least a portion of the top surface may be angled or and/or not flat or planar. The angled top surface portion may be positioned at the anterior end of the superior base. The angled top surface portion may be positioned at the medial and/or the lateral ends. The angled top surface portion may be positioned at the anterior end, at the medial end and the lateral end. At least a portion of the top surface contacts the vertebra, endplate or bone. At least a portion of the top surface contacts the endplate of a vertebra and/or the endplate of the upper vertebra. The superior base further may comprise a keel, a flange and a posterior wall or posterior tab.

In one embodiment, the superior base comprises a keel and/or an upper keel 270. The upper keel can include a height and a length. The upper keel desirably extends upwardly from the superior base and/or extends upwardly from the top surface of the superior base. The upper keel may extend orthogonally or perpendicular to the superior base and/or may extend orthogonal or perpendicular to a top surface of the superior base. At least a portion of at least one surface of the upper keel desirably contacts the vertebra. At least a portion of the at least one surface of the upper keel desirably contacts the endplate of a vertebra and/or the endplate of the upper vertebra. At least a portion of the at least one surface desirably contacts cancellous and/or cortical bone.

The length of the upper keel may extend from the posterior end or second end towards the first end or anterior end. The length of the upper keel extends from the posterior end or second end towards the first end or anterior end and extends upwardly away from the superior base and/or the top surface of the superior base. The length of the upper keel may match or substantially match a length of the superior base and/or the upper keel may match or substantially match the length of a top surface of the superior base. At least one or more surfaces of the upper keel contacts the vertebra bone and/or at least one or more surfaces of the upper keel contacts the cancellous bone and/or cortical bone of the vertebra and/or the cancellous bone and/or cortical bone of the upper vertebra. The upper keel comprises a shape. The shape includes a shape substantially similar to a trapezoid, trapezium, rhombus, parallelogram and/or a sloped rectangle. The first end or anterior end of the upper keel can optionally be sloped or at an angle to facilitate easier positioning and/or atraumatic insertion. In another embodiment, the length may comprise or function as an additional structural support component to the superior base, including acting similar to structures such as a truss, I-beam or H-beam. Such structural components may be helpful in supporting the superior base to provide a more rigid structure, resist bending and/or resist shear when coupled to the posterior wall or tab. Accordingly, such structural components may assist with supporting the superior base to provide a more rigid structure within the centroid region due to the thinner height during motion, as well as resist bending and/or resist shear when coupled to the posterior wall or tab.

The superior base can further include a flange 280 which can extend from or otherwise be spaced apart from the bottom surface 260 of the superior base to create a recessed channel 290 which can be sized and/or configured to receive a portion of the superior articulating element and/or the ASP. The flange comprises a flange width and may optionally include an overhanging portion or lip 295, wherein the flange width is sized and configured to be disposed into a portion of the superior articulating element. In various embodiment the flange can comprise a smaller width than the width of superior base. Alternatively, the first contacting surface comprises a larger width than the flange width. The first contacting surface may extend beyond the flange. The flange substantially surrounds the perimeter of the superior base, with various portions of the flange recessed and/or removed. The flange width is sized and configured to be disposed into a gutter or channel of the superior articulating element. The flange width is sized and configured to capture the ASP or preformed ASP and prevent any unwarranted migration. The recessed channel is sized and configured to receive a portion of the ASP or the preformed ASP by allowing the flow of the ASP during insert or over molding and/or compression molding, desirably creating a mechanical interlock or bond between the underlying base and the over molded polymer material.

The superior base further can optionally comprise a posterior wall or tab 300, which may be coupled or integrally formed to the upper keel and/or the superior base. The posterior wall or tab can be positioned on the second end or posterior end of the superior base. The posterior wall extends upwardly to extend past the top surface of the superior base. The posterior end of the upper keel intersects with the anterior facing surface of the posterior wall or tab. The posterior end of the upper keel intersects orthogonally or perpendicularly with the anterior facing surface of the posterior wall or tab. At least a portion of the anterior facing surface of the posterior wall or tab may contact bone and/or at least a portion of the anterior facing surface of the posterior wall or tab contacts the posterior facing surface of the vertebra and/or the upper vertebra. The posterior wall or tab can desirably function as a positive stop limiter or provide tactile feedback to surgeons for proper placement of the superior element between the upper and lower vertebra and/or within the disc space. Desirably this structure may prevent the superior element from migrate anteriorly in an unwanted manner during placement and/or long-term use. The posterior wall may include an anterior facing surface and a posterior facing wall that is flat or planar. The posterior wall or tab may include an anterior facing surface and a posterior facing wall that is not flat or not planar. Furthermore, the position of the posterior wall or tab may be monitored with fluoroscopy or other visualization methods during surgery to determine the progress of the implantation and to confirm when the superior element has been correctly implanted-such as by providing confirmation that the posterior wall or tab contacts and/or is recessed against a posterior wall of the vertebral body or the upper vertebral body.

In another embodiment, the superior base may comprise an instrument opening 310. The instrument opening can be sized and configured to receive an instrument and/or at least a portion of an implantation instrument. The instrument opening may be uniform or non-uniform. The instrument opening may include a conical shape. Instruments that may be received include a driver, a deployment tool, and/or any tool that can be inserted within the instrument opening to push and/or slide the superior element to the proper positioning between the upper and lower vertebral bodies. The instrument opening may be tapered and/or at least a portion of the instrument opening may be tapered.

In another embodiment, the superior base may comprise a second contacting surface 320. The second contacting surface may be an inferior facing surface which is sized and configured to receive a portion of the ASP. The second contacting surface of the superior base is recessed from the flange and/or the second contacting surface of the superior base is below the inferior facing surface of the flange. In another embodiment, the superior base comprises a socket with a third contacting surface. The third contacting surface comprises a shape, the shape includes a hemispherical shape, a convex shape, arch shape, a dome shape and/or any combination thereof. The third contacting surface is sized and configured to receive a portion of the ASP.

As defined herein, a “spherical” shaped surface could include any curved surface having a uniform radius of curvature and may refer to a spherical cap or a segment of a sphere. In various alternative embodiments, non-spherical curved surfaces may function as articulation surfaces to impart specific limits to the range of motion of the prosthetic device. In still another alternative embodiment, the joint may be inverted with the upper articulation surface having a convex shape and the lower articulation surface having a concave articulation surface.

In various embodiments, the superior base may comprise a material including, but not limited to, metal, polymers or ceramic and/or any combination thereof. The metals may comprise titanium, titanium alloys, cobalt-chrome alloys, platinum, stainless steel and/or any combination thereof. More specifically, the metal includes titanium and/or cobalt-chrome molybdenum (CoCrMo). The polymers may include thermoplastic or thermoset polymers. The polymers may further include carbon fiber, polyether ether ketone (PEEK), polyethylene (PE), ultra-high molecular weight polyethylene (UHMWPE), polycarbonate (PC), polypropylene (PP) and/or any combination thereof. The ceramics may include alumina ceramics, Zirconia (ZrO2) ceramics, Calcium phosphate or hydroxyapatite (Ca10(PO46(OH)2) ceramics, titanium dioxide (TiO2), silica (SiO2), Zinc Oxide (ZnO) and/or any combination thereof. The materials may be manufactured using traditional methods and/or using 3D printed techniques known in the art. Furthermore, the material may comprise a porous material, the porous material including (but not limited to) porous metal, porous polymer, porous ceramic and/or any combinations thereof.

In another embodiment, at least one surface of the superior base may comprise a coating and/or surface texture to desirably help facilitate healing or osseointegration, and/or to better accommodate loading forces and/or wear, such as shown in FIG. 2H. Alternatively, at least one or more surfaces of the superior base comprises a coating and/or surface texture. At least a portion of the top surface of the superior base comprises a coating (not shown) and/or a surface texture or surface finish. At least a portion of the first contacting surface comprises a coating and/or surface texture. At least a portion of the second contacting surface comprises a coating and/or surface texture. At least a portion of the third contacting surface comprises a coating and/or surface texture. Accordingly, the coating and/or surface texture disposed onto each of the one or more top surfaces, the first contacting surface, the second contacting surface, and/or the third contacting surfaces of the superior base may be the same surface texture. the coating and/or surface texture disposed onto each of the one or more top surfaces, the first contacting surface, the second contacting surface, and/or the third contacting surfaces of the superior base may be different.

The surface textures or finishes may comprise threads, flutes, grooves, and/or teeth that may include various shapes. The various shapes may include tapered, stepped, conical and/or paralleled, flat, pointed, and/or rounded. The surface textures or finishes may further comprise roughened surfaces or porous surfaces, including turned, blasting, sand blasting, acid etching, chemical etching, dual acid etched, plasma sprayed, anodized surfaces, and/or any combination thereof. The surface textures or finishes may further include a polish surface finish or texture. The polished surface may be accomplished using different techniques, mechanical polishing, chemical polishing, electrolytic polishing, and/or any combination thereof. Polished surfaces can be measured in “Ra” micrometers (μm) or microinches (μin.). The Ra may comprise a range of 0.025 to 1.60 μm; may comprise a range of 0.025 to 0.30 μm; may comprise a range of 0.025 to 0.20 μm; may comprise a range of 0.025 to 0.10 μm; and/or may comprise a range of 0.05 to 0.20 μm. Accordingly, the Ra may comprise at least 0.05 μm or higher; at least 0.10 μm or higher and/or at least 0.8 μm or higher. Surface structure is often closely related to the friction and wear properties of a surface. A surface with a large Ra value will usually have somewhat higher friction and wear quickly, and a surface with a lower Ra value will have a lower friction and enhanced part performance and/or prevent or reduce unwanted adhesion of molecules or components to surface(s) (e.g., surfaces are smooth, shiny and less porous). A polished surface has many further advantages, including improving cleanability, increases resistance to corrosion, reduces adhesive properties (for cells or other blood components to attach to), increases biocompatibility, increased light reflection for enhanced radiopacity, etc.

The coatings may include inorganic coatings or organic coatings. The coatings may further include a metal coating, a polymer coating, a composite coating (ceramic-ceramic, polymer-ceramic, metal-ceramic, metal-metal, polymer-metal, etc.), a ceramic coating, an anti-microbial coating, a growth factor coating, a protein coating, a peptide coating, an anti-coagulant coating, an antioxidant coating and/or any combination thereof. The antioxidant coatings may comprise naturally occurring or synthetic compounds. The natural occurring compounds comprises Vitamin E and Vitamin C (tocotrienols and tocopherols, in general), phenolic compounds and carotenoids. Synthetic antioxidant compounds include α-lipoic acid, N-acetyl cysteine, melatonin, gallic acid, captopril, taurine, catechin, and quercetin, and/or any combination thereof. The coatings can be impregnated, applied and/or deposited using a variety of coating techniques. These techniques include sintered coating, electrophoretic coating, electrochemical, plasma spray, laser deposition, flame spray, biomimetic deposition and wet methods such as sol-gel-based spin-and-dip or spray-coating deposition have been used most often for coating implants.

The metal coatings may comprise titanium, titanium alloys, cobalt-chrome alloys, platinum and stainless steel, and/or any combination thereof. More specifically, the metal coating includes titanium and/or cobalt-chrome molybdenum (CoCrMo). The polymer coatings may include thermoplastic or thermoset polymers. The polymers may further include carbon fiber, polyether ether ketone (PEEK), polyethylene (PE), ultra-high molecular weight polyethylene (UHMWPE), polycarbonate (PC), polypropylene (PP) and/or any combination thereof. The ceramic coatings may include alumina ceramics, Zirconia (ZrO2) ceramics, Calcium phosphate or hydroxyapatite (Ca10(PO46(OH)2) ceramics, titanium dioxide (TiO2), silica (SiO2), Zinc Oxide (ZnO) and/or any combination thereof. In one embodiment, the superior base comprises a Titanium coating. The titanium coating was deposited using the plasma spray technique.

In another embodiment, the first processing may include any methods known in the art to consolidate or compress the antioxidant stabilized polymer (ASP) into a suitable form, molded ASP or preformed ASP for use as or as part of a preformed implant, a final implant, a preformed articulation component and/or a final articulation component. Suitable consolidation or compression methods comprises compression molding, direct compression molding, blow molding, extrusion molding, injection molding, hot isostatic pressing, ram extrusion, rotational molding, and/or any combination thereof. The first processing may further include annealing, curing, cooling, and/or any combination thereof.

In another embodiment, the first processing may further include insert molding or over molding. Insert molding or over molding allows a seamless combination of multiple materials, parts or components into a single implant and/or a portion of an implant. Insert molding or over molding allows a part, a component or base element to be inserted into the mold prior to injecting or incorporating the ASP to create a mold assembly. Once the component or base element is properly positioned within the mold, the ASP or molten ASP is incorporated into the mold assembly under high pressures to fill the entire mold assembly to ensure the ASP adheres completely to the component or base element. Such a process can increase mechanical properties of polymers and/or the ASP, reduce assembly cost, decreases fixation components of the ASP to the base element, and/or decreases potential misalignment, improper terminations, loosening, migration, etc.

In one exemplary embodiment, the first processing comprises direct compression molding (DCM). The DCM mold may contain a release agent or may be used without a release agent. The DCM mold may be filled with the antioxidant stabilized polymer (ASP). The DCM mold is sealed or closed to consolidate and/or compress the ASP into the desired preformed articulation component, final articulation component, portion of a final implant or final implant. The mold is then heated and pressurized for a set time to complete the DCM process. Once the desired compression of the preformed articulation component, final articulation component, portion of a final implant or final implant is completed, it may be subject to additional processes, including annealing, curing and/or cooling. Once cooled, the preformed articulation component or final articulation component can be removed from the DCM mold. Annealing is the process of heating polymers in temperature ranges that fall below or above the material's glass transition temperature and/or melt temperature to eliminate internal pressures and/or stresses on the material.

In another exemplary embodiment, the first processing comprises direct compression molding (DCM) and over molding or insert molding. The DCM mold may further comprise a part or substrate that the antioxidant polymer blend will be coupled to, over molded with and/or compressed against. The part, component or base element comprises a portion of a final implant or a final implant, the part or substrate comprises a superior base. The DCM mold may be filled with the antioxidant polymer blend. The DCM mold is sealed or closed to compress the antioxidant polymer blend over the part or substrate, such that a portion of the antioxidant polymer blend flows in multi-directions to conform to a desired shape or locking features of the part or substrate to create the desired preformed articulation component, final articulation component, a portion of a final implant or final implant. Such over molding and compression molding over a part or substrate facilitates or enhances the coupling or bond between the two components—i.e., the antioxidant polymer blend and the part or substrate. Once the desired compression of the preformed articulation component, final articulation component, portion of a final implant or final implant may be subject to additional processes, including annealing, curing and/or cooling. Once cooled, it is removed from the DCM mold. Annealing is the process of heating polymers in temperature ranges that fall below or above the material's glass transition temperature to eliminate internal pressures and/or stresses on the material.

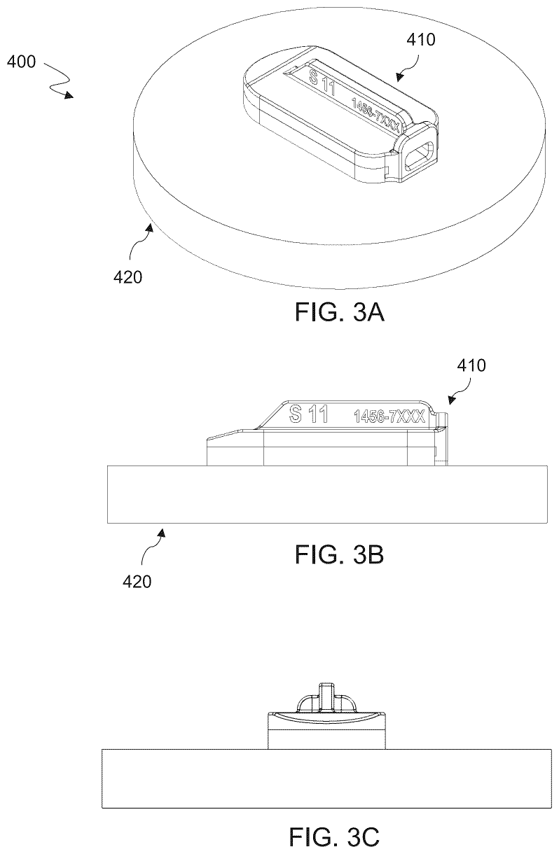

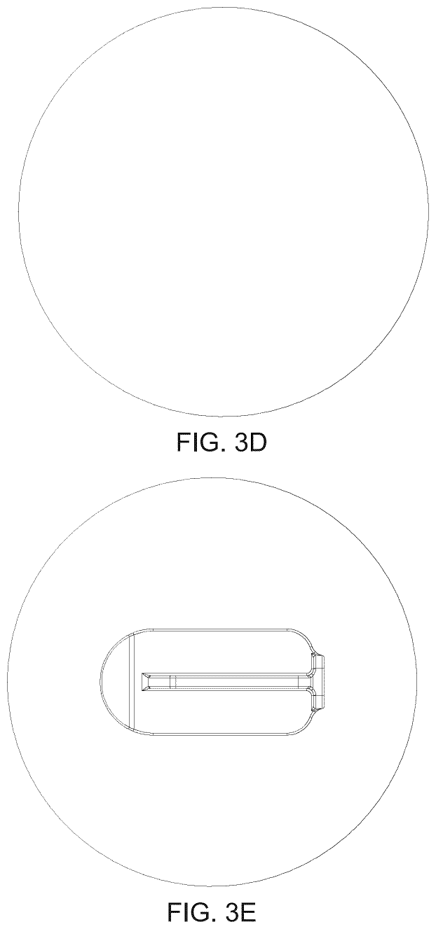

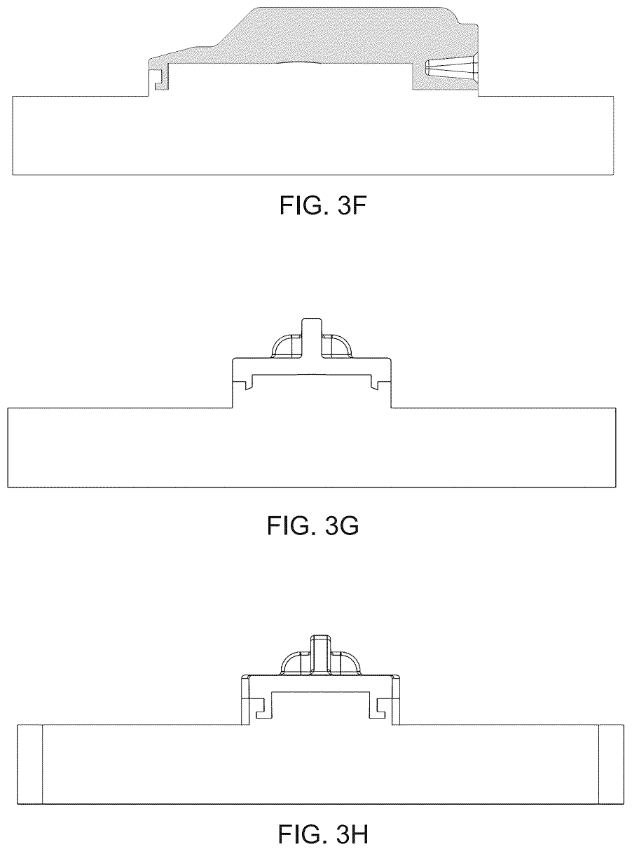

Once the first processing is completed, a preformed implant can be created. FIGS. 3A-3E illustrate various views of one exemplary embodiment of a preformed implant and/or a portion of a preformed implant. The preformed implant 400 comprises a base element 410 and a preformed or molded ASP 420. Alternatively, the preformed implant may comprise a preformed or molded ASP. The preformed implant and/or a portion of a preformed implant comprises a semi-finished part or product. The term “preformed” as used herein means ASP's or polymer materials that can be consolidated, compressed and/or otherwise formed, either alone and/or in conjunction with a base element to form a generic, semi-finished and/or finished part that is shaped into a bar, rod, sheet, cylinder, slab, or the like, and which may require further processing to obtain a final implant or final articulation component.

The preformed or molded ASP comprises a top surface, a bottom surface, a diameter and a thickness. At least a portion of the top surface and a portion of the bottom surface is flat or planar. At least a portion of the top surface and a portion of the bottom surface is flat or planar and/or uniform. The diameter comprises 50 mm or less; the diameter comprises 46 mm or less; the diameter comprises at least 40 mm or greater. The thickness of the preformed or molded ASP comprises less than or equal to 4 cm (1.575 in). The thickness comprises 0.333 in or less and/or the thickness comprises 0.25 in or less. The thickness of the preformed or molded ASP is less than or equal to 4 cm and is uniform. The dimensional characteristics, the surfaces, thickness and/or diameter, of the preformed or molded ASP results in the uniformity of the cross-linking across the surface area and the thickness of the preformed or molded ASP.

In another embodiment, the method of forming a cross-linked stabilized spinal implant may comprise the step of completing a first crosslinking of the preformed implant with a selected first crosslinking technique; and/or completing a second crosslinking the final implant with a selected second crosslinking technique. The selected techniques include a chemical technique, gas technique or an ionizing or irradiating technique. Polymer or ASP grafting, crosslinking and/or sterilization can be achieved by chemical or irradiation techniques. The grafting, crosslinking and sterilization requires additives to initiate the reactions for chemical techniques. Using irradiation techniques does not require additives for the reactions, can be used at any temperature range, and may have a greater penetration depth, for well controlled grafting and crosslinking. Accordingly, crosslinking using an irradiation technique allows high energy electrons to bombard the preformed implant, final articulation component or final implant. The energy of irradiation ejects a hydrogen atom which then removes a neighboring hydrogen atom, forming molecular hydrogen gas (H2), which may affect the crystallinity and oxidation of polymers. The irradiation technique or ionizing technique may comprise alpha radiation, x-ray photons, gamma beam or gamma irradiation, cold-gamma beam irradiation, e-beam (electron beam) irradiation, and/or any combination thereof. The gas technique may comprise gas plasma, ethylene oxide (ETO) and/or any combination thereof. In one exemplary embodiment, the irradiation technique comprises gamma beam. Multiple factors may influence the crystallinity and oxidative degradation of the polymer, the ASP and/or the preformed ASP when selecting the desired irradiation technique. The irradiation technique comprises factors such as a radiation dose, a dose rate, a controlled environment and/or any combination thereof.

In one embodiment, the irradiation technique comprises a radiation dose and/or a dose rate. As described herein, the dose and/or the dose rate may influence the crystallinity and oxidation of a polymer. More specifically, at higher doses, the transvinylene content and the crosslink density may increase. The radiation dose should be optimized to obtain a desired crosslinking or wear rate and the reduction of oxidation of the polymer material. The irradiation dose may comprise at least 5 kGy or greater; the dose of at least 10 kGy or greater; and/or an irradiation dose of at least 20 kGy or greater. The gamma beam dose may comprise 20 kGy to 100 kGy; may comprise an irradiation dose of 20 kGy to 60 kGy; and/or may comprise an irradiation dose of 45 kGy to 55 kGy. The irradiation can be carried out in a controlled environment, the controlled environment may comprise a desired atmosphere, the desired atmosphere includes air atmosphere, insert atmosphere and/or vacuum atmosphere. The air atmosphere may include oxygen, wherein the oxygen concentration in the atmosphere is at least 1%, 2%, 4%, or up to about 22%, or any value thereabout or therebetween. The inert atmosphere includes a gas selected from the group consisting of nitrogen, argon, helium, neon, or the like, or a combination thereof. Crosslinking in oxygen depleted environments or inert atmosphere can reduce the degree of oxidative degradation.

The controlled environment may further comprise a radiation temperature. The radiation temperature includes room temperature or ambient temperature, a temperature below the melting point of the polymeric material, a temperature above the melting point of the polymeric material, a temperature below the glass transition temperature of the polymeric material, a temperature above the glass transition temperature of the polymeric material, and/or any combination thereof. The irradiation can be carried out at any temperature or at any dose rate using e-beam, gamma, and/or X-ray.

The controlled environment may optionally further comprise annealing the crosslinked preformed implant or the cross-linked preformed ASP. This process can melt some or all of the crystalline regions and allow the recombination of the trapped free radicals in these regions. Once, the polymer is re-crystallized, the residual free radicals will desirably have been eliminated or substantially eliminated and this wear resistant polymer, the ASP or the preformed ASP is also oxidatively stable. Another annealing method comprises heating below the melting point after radiation cross-linking or heating above the melting point of the polymeric material, the ASP or preformed ASP. Annealing may occur in ambient atmosphere, a vacuum atmosphere or an inert atmosphere.

In another embodiment, the method of forming a cross-linked stabilized spinal implant may comprise a step of packaging the preformed implant and/or a final implant in a packaging container in a controlled environment. The packaging step may occur prior to a first crosslinking and/or after crosslinking. The packaging containers may comprise a gas permeable packaging, a multi-layer polymeric barrier packaging, a metallic foil packaging, a combination of polymeric and foil packaging, and/or any combination thereof. The gas permeable packaging comprises double PET blister with Tyvek cover. The multi-layer polymeric film barrier packaging may comprise PE/PA multi-layer, a PE/PET/PVOH multi-layer, and/or a PE/PET/PE multi-layer package. The barrier packaging may comprise a PET blister and an AL foil cover, a Al foil pouch inside a PET blister or a PE/PET/PE multi-layer. The metallic foil packaging may include metal foil pouches such as aluminum or MYLAR® polyester coated packaging foil which is available commercially for heat-sealed vacuum packaging. Polymeric packaging materials such as polyethylene terephthalate and polyethylene vinyl alcohol), both of which are commercially available may also be used.

Other controlled environment factors such as packaging atmosphere and packaging processing conditions may also influence the distribution and the amount of the oxidation products. The controlled environment may include a desired atmosphere, desired temperature. The desired atmosphere includes air atmosphere, insert atmosphere and/or vacuum atmosphere. The air atmosphere may include oxygen, wherein the oxygen concentration in the atmosphere is at least 1%, 2%, 4%, or up to about 22%, or any value thereabout or therebetween. The inert atmosphere includes a gas selected from the group consisting of nitrogen, argon, helium, neon, or the like, or a combination thereof. Packaging in oxygen depleted environments or inert atmosphere can reduce the degree of oxidative degradation. Furthermore, other parameters of controlled environments, such as radiation temperature, annealing, packaging atmosphere and/or packaging type may influence the distribution and the amount of the oxidation in the preformed implant and/or final implant.

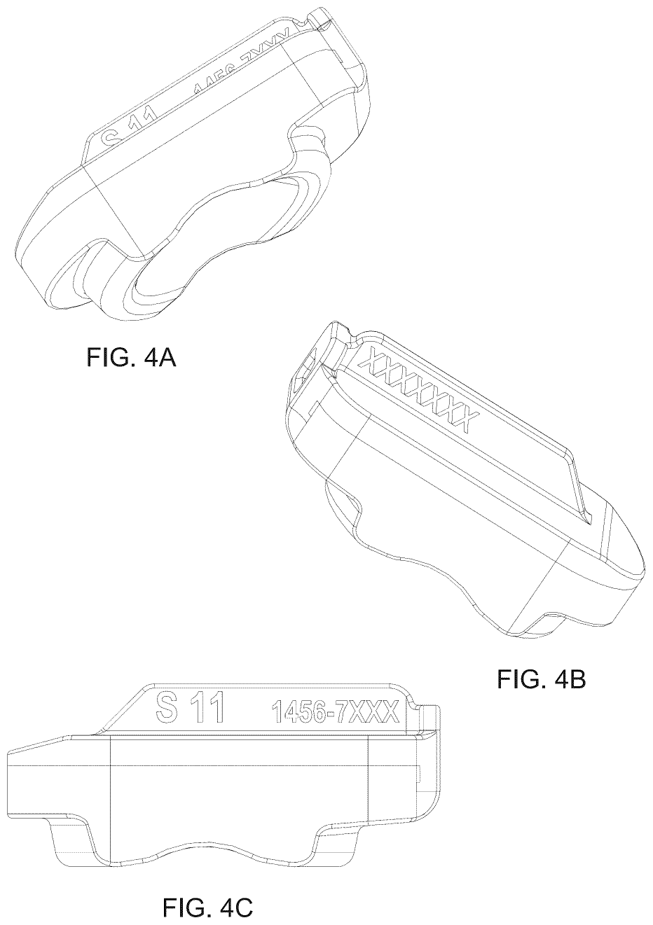

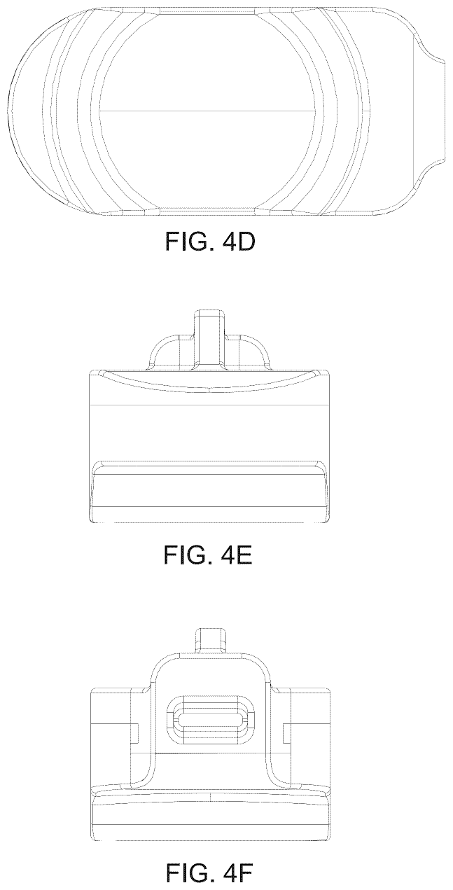

In another embodiment, the method of forming a cross-linked stabilized spinal implant may comprise a step of completing a second processing of the preformed implant to create a final implant. The second processing may comprise various manufacturing methods known in the art that help facilitate further processing on the preformed articulation component, final articulation component and/or final implant. Such methods include milling, machining, drilling, cutting, surface finishing, deburring, assembling with other components, with or without lubricants and/or other manufacturing methods to create final contact surfaces, surface geometries and/or surface roughness. In one exemplary embodiment, the second processing may comprise CNC milling and turning in a separate controlled environment to create the final implant and/or a portion of a final implant as shown in FIGS. 4A-4F. The portion of the final implant or the final implant may comprise a superior base and a final or formed ASP.

In another embodiment, the method of forming a crosslinked stabilized spinal implant comprises the step of: packaging the final implant, the final implant and/or the final implant assembly. Packaging is generally carried out using either gas permeable packaging or barrier packaging. The packaging may comprise a reduced oxygen atmosphere or vacuum. The packaging may also comprise an air, gas plasma, and/or an inert gas backfill comprising argon, nitrogen, and/or an oxygen scavenger. In one exemplary embodiment, the packing comprises a barrier packaging with a vacuum seal (reduced or eliminated oxygen environment) and an inert gas flush prior to sterilization. The inert gas flush comprises nitrogen.

In another embodiment, the method of forming a cross-linked stabilized spinal implant may comprise the step of completing a second crosslinking and sterilization of the final implant or the final implant with a selected second cross-linking technique in a second controlled environment. The selected techniques include a chemical technique, gas technique or an ionizing or irradiating technique. Accordingly, crosslinking using an irradiation technique allows high energy electrons to bombard the preformed implant, final articulation component or final implant. The energy of irradiation ejects a hydrogen atom which then removes a neighboring hydrogen atom, forming molecular hydrogen gas (H2). The irradiation technique or ionizing technique may comprise gamma beam or gamma irradiation, cold-gamma beam irradiation, e-beam irradiation, and/or any combination thereof. The gas technique may comprise gas plasma, ethylene oxide (ETO) and/or any combination thereof. In one exemplary embodiment, the irradiation technique comprises gamma beam. Multiple factors may influence the crystallinity and oxidative degradation of the polymer, the ASP, the preformed or molded ASP, the preformed implant, the final implant and/or any combination thereof when selecting the desired crosslinking technique. The technique comprises factors such as a radiation dose, a dose rate, a controlled environment and/or any combination thereof.

In one embodiment, the second crosslinking technique comprises a radiation dose. The radiation dose should be optimized to obtain a desired crosslinking or wear rate and the reduction of oxidation of the polymer material, the preformed ASP, the formed ASP, the preformed implant and/or the final implant. The irradiation dose may comprise at least 5 kGy or greater; the dose of at least 10 kGy or greater; and/or an irradiation dose of at least 20 kGy or greater. The gamma beam dose may comprise 20 kGy to 100 kGy; may comprise an irradiation dose of 20 kGy to 60 kGy; and/or may comprise an irradiation dose of 45 kGy to 55 kGy. The irradiation can be carried out in a controlled environment, the controlled environment may comprise a desired atmosphere, the desired atmosphere includes air atmosphere, insert atmosphere and/or vacuum atmosphere. The air atmosphere may include oxygen, wherein the oxygen concentration in the atmosphere is at least 1%, 2%, 4%, or up to about 22%, or any value thereabout or therebetween. The inert atmosphere includes a gas selected from the group consisting of nitrogen, argon, helium, neon, or the like, or a combination thereof.

The controlled environment may further comprise a radiation temperature. The radiation temperature includes room temperature or ambient temperature, a temperature below the melting point of the polymeric material or ASP, a temperature above the melting point of the polymeric material, a temperature below the glass transition temperature of the polymeric material, a temperature above the glass transition temperature of the polymeric material, and/or any combination thereof. The irradiation can be carried out at any temperature or at any dose rate using e-beam, gamma, and/or X-ray.

The controlled environment may further comprise annealing the crosslinked preformed implant, the cross-linked preformed ASP, formed or final implant, and/or formed or final ASP. This melts the crystalline regions and allows the recombination of the trapped free radicals in these regions. Once, the polymer is re-crystallized, the residual free radicals have been eliminated or substantially eliminated and this wear resistant polymer, the ASP or the preformed ASP is also oxidatively stable. Another annealing method comprises heating below the melting point after radiation cross-linking or heating above the melting point of the polymeric material, the ASP, preformed ASP, formed ASP, preformed implant and/or final implant.

The sterilization may be accomplished during the crosslinking step, or as part of a separate processing step, and/or as a second cross-linking step. The sterilization may comprise a same or different radiation dose, a dose rate, a controlled environment and/or any combination thereof compared to the first crosslinking step. Alternatively, the sterilization may comprise a same or different radiation dose, a dose rate, a controlled environment and/or any combination thereof compared to the second crosslinking step. The first crosslinking step may comprise a same or different radiation dose, a dose rate, a controlled environment and/or any combination thereof compared to the second crosslinking step.

### Specific Embodiments

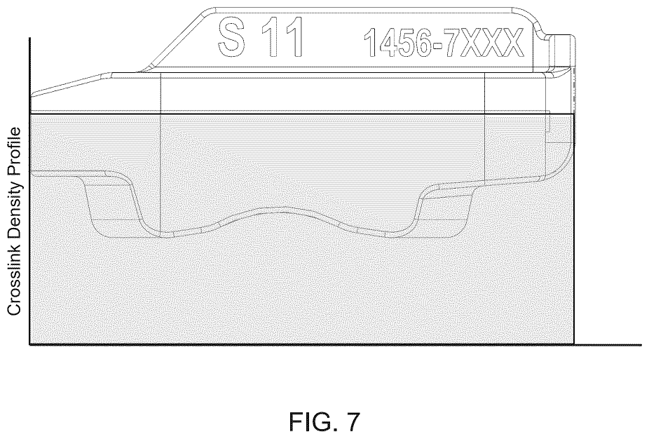

Various embodiments disclosed herein comprise a polymer material which is combined with an antioxidant (e.g., at a preferred concentration/dispersion range of 900 ppm to 1100 ppm or greater of antioxidant) to create a polymer/antioxidant blend (the “ASP”). The ASP may be consolidated onto a superior base element to fabricate into a desired preformed implant. The preformed implant comprises a superior base element and a preformed, molded, unfinished ASP (e.g., a blank or stock). The preformed, molded, unfinished ASP comprises a uniform thickness of 0.25 in or less and/or a uniform and planar surface area to allow for optimal and uniform crosslink density and crystallinity. The preformed implant can be subjected to a first crosslinking irradiation. The crosslinked preformed, unfinished, molded implant may then be subjected to a second processing (e.g., machining) to create the final implant, as well as subsequent packaging and a second crosslinking. By having the final implant undergo multiple crosslinking steps, a unique crosslink profile is created throughout the final implant morphology. The crosslink profile may change along the thickness or height of the final implant. The crosslink profile may include crosslink density and/or crystallinity. The crosslink profile allows the final implant to have a mechanical property profile that changes similarly. Of course, in various alternative embodiments, such as shown in FIG. 7, an implant having a uniform cross-linked profile throughout the depth of the implant may utilized in the various methods and steps described herein.

In one embodiment, the preformed implant comprises a superior base element and a preformed, molded, unfinished ASP. The molded, unfinished ASP comprises a polymer and a stabilizer. In one exemplary embodiment, the polymer comprises UHMWPE and the stabilizer comprises an antioxidant, the antioxidant comprises Vitamin E (e.g., α-tocopherol, α-tocopherol derivatives). The preformed, molded, unfinished ASP may further comprise suitable additives that include radiopaque materials, antimicrobial materials such as silver ions, antibiotics, and microparticles and/or nanoparticles serving various functions. Preservatives, colorants and other conventional additives may also be used.

UHMWPE are thermoplastics of polyethylene, with extreme long carbon-chains and molecular weights. The molecular weight and its distribution can be controlled by process parameters such as temperature, time and pressure. UHMWPE generally has a molecular weight of at least between 2 and 6 million. The fracture toughness, low friction coefficient, high impact strength, and low density of UHMWPE have made it a popular choice as the articulating surfaces of joint replacements, such as hip, knee, ankle, shoulder and now, spine. The synthesis of UHMWPE results in raw materials in the form a resin, a powder, pellets, flakes, particles, and/or any combination thereof.

Suitable stabilizers generally include materials that can be added in an effective amount to the UHMWPE material to, at least in part, inhibit the oxidation cycle caused by irradiation of UHMWPE. A suitable stabilizer comprises an antioxidant, the antioxidant includes Vitamin E. As used herein “vitamin E” comprises derivatives of α-tocopherol and/or α-tocopherol. Advantageously, by incorporating an antioxidant, such as α-tocopherol, into the UHMWPE prior to subjecting the preformed, molded or unfinished ASP to crosslinking irradiation, the UHMWPE may be stabilized without the need for post irradiation melt annealing or any other post-irradiation treatment to quench free radicals. Specifically, an antioxidant, such as α-tocopherol, acts as a free radical scavenger and, in particular, acts as an electron donor to stabilize free radicals. While α-tocopherol itself then becomes a free radical, tocopherol is a stable, substantially unreactive free radical. Additionally, because of the substantially reduced level of oxidation that occurs using a UHMWPE/antioxidant blend, the amount of oxidized material that must be removed to form a final implant is reduced. As a result, the size of the stock material subjected to irradiation may be smaller in dimension, making it easier to handle and easier to manufacture into final medical implants.

Desirably, the antioxidant concentration/dispersion may comprise a value between 0.01 weight percent (wt. %) and 3 wt. %, and/or one preferred range of 900 ppm to 1100 ppm or greater of antioxidant to the remaining polymer constituents. In exemplary embodiments, the antioxidant concentration may be as low as 0.01 wt. %, 0.05 wt. %, and 0.1 wt. %, or as high as 0.6 wt. %, 0.8 wt. %, and 1.0 wt. %, for example. In determining the appropriate amount of antioxidant, two competing concerns exist. Specifically, the amount selected should desirably be high enough to quench free radicals in the UHMWPE, but should also desirably be low enough to allow sufficient crosslinking so as to maintain acceptable wear properties of the UHMWPE. In one exemplary embodiment, a range of the antioxidant may comprise a concentration from 0.05 wt. % to 0.3 wt % is used, and more preferably approximately 0.1 wt % is used, to successfully quench free radicals while still maintaining acceptable wear properties.

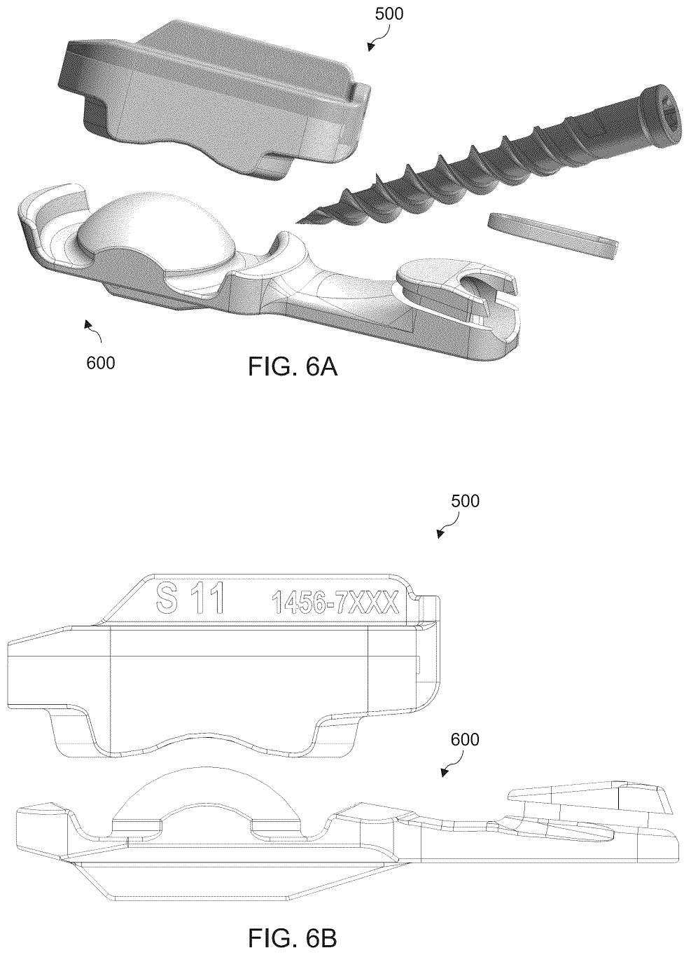

Once the ASP is formed, it may be processed to consolidate the ASP into a preformed or unfinished ASP. The consolidation step may include direct compression molding (DCM) and insert molding. The consolidation step or process creates or forms the preformed implant, which includes the ASP and a superior base element. The preformed or unfinished ASP refers to a molded or unfinished material that is suitable for crosslinking. In various embodiments, a plurality of preformed implants can desirably be manufactured with differing sizes and/or shapes, For example, a kit of final implant components could include a plurality of superior components, with each superior component including an finished or final ASP component of similar size and/or shape that is secured to a superior element base component having differing size and/or shapes (e.g., size 11, 12, 13, 14 and 15 implants), resulting in a series of superior components having varying cephalad (superior)/caudad (inferior) thicknesses as shown in FIG. 6A-6B. Desirably, each of these different sized/shaped superior final implant components can be utilized in combination with a single size of caudal or inferior final implant component, thereby significantly reducing the number of caudal or inferior final implant components (and overall number of individual components) required for an individual surgical kit to accommodate anatomical variation and/or surgical objectives for a targeted patient population. Similarly, a surgical kit could comprise a plurality of superior and/or inferior implant components having differing longitudinal lengths, such as small, medium and large implant, which desirably accommodates anatomical variation and/or surgical objectives for a targeted patient population. Such components could be manufactured in a similar fashion as previously described, with these components desirably able to function with a single size (e.g., small, medium or large) caudal or inferior component.

The preformed, molded or unfinished ASP may undergo a first crosslinking using a first crosslinking technique in a first controlled environment. The first crosslinking technique comprises gamma irradiation. The first crosslinking technique comprises a first irradiation dose and a first dose rate. The first irradiation dose rate comprises 40 to 60 kGy; and/or 45 to 55 kGy. The irradiation may be performed in air at atmospheric pressure, in a vacuum chamber at a pressure substantially less then atmospheric pressure, or in an inert environment, i.e., in an argon environment, for example.

After the first crosslinking, the molded, unfinished or preformed implant can be subjected to a second processing to create a finished implant component. The second processing types include (but not limited to) further processing or manufacture steps such as milling, machining, drilling, cutting, polishing, assembly with other components, and/or other manufacturing or pre-manufacturing steps conventionally employed to manufacture implants. After such second processing, the finished implant may undergo a second crosslinking using a second crosslinking technique in a second controlled environment. The finished implant may undergo a second crosslinking using a second crosslinking technique in a second controlled environment. The second crosslinking technique comprises gamma irradiation. The second crosslinking technique comprises a second irradiation dose and a second dose rate. The second irradiation dose rate comprises 20 to 30 kGy; and/or 15 to 35 kGy. The second crosslinking technique may comprise the same crosslinking technique as the first crosslinking technique. The second crosslinking technique may further comprise sterilization of the final implant.

In various embodiments, some level of selective shielding may occur which protects certain areas of the preformed implant and/or the final implant from exposure to irradiation by the gamma irradiation. In one embodiment, portions of the superior base element can partially and/or fully shield regions of the preformed, molded, unfinished ASP and/or the final ASP attached thereto from some levels of the crosslinking radiation, which can allow the preformed, molded, unfinished ASP and/or the final ASP in those regions to retain a higher level of ductility and/or flexibility as compared to preformed, molded, unfinished ASP and/or the final ASP in other regions (such as on the articulating and/or “bumper” surfaces), thus better securing the preformed, molded, unfinished ASP and/or the final ASP to the underlying metal component in a desired manner.

Moreover, such selective shielding and/or multiple crosslinking steps can also create a crosslink density and/or crystallinity profile that changes along the thickness or depth of the preformed or final ASP. Alternatively, such selective shielding and/or multiple crosslinking steps can also create a crosslink density and/or crystallinity profile that changes from the inferior facing surface towards the junction between the superior base element and/or preformed or final ASP. Therefore, by selecting the gamma crosslinking irradiation technique and optimally controlling dose, dose rate, radiation source and its half-life, morphology (e.g., uniform or non-uniformity) and thickness of polymer (preformed ASP or final ASP), it plays an essential role in the enhanced properties of the preformed or final ASP and creates a final implant with optimal wear resistance.

Crosslinking agents tie together carbon atoms from different chains of the polymer, transforming what were once viscous linear segments into an insoluble gel network that no longer melts or flows like a typical thermoplastic. The degree of crosslinking that occurs is determined by the percentage of polymer chains that are interconnected in this network, or in other words, the density. Also, the degree of crosslinking depends upon polymer structure (e.g., polymer surface area and/or polymer thickness), phase morphology, irradiation of gamma radiation with controlled dose and duration, and nature of gamma radiation source. Higher crosslink density is the result of more linkages per length of polymer chain, resulting in larger property changes. Because crosslinking prevents molecules from slipping by each other in the amorphous regions of the resin, it especially provides various advantages, such as: (1) higher long-term service temperatures, including better heat and dimensional stability (and as little as 1% the creep of the non-crosslinked polymer); (2) improved impact resistance and environmental stress-cracking resistance (ESCR); (3) higher tensile strength and stiffness properties, especially at high temperatures; (4) better “shape memory” or “memory effect,” in which a heated and deformed crosslinked material holds its shape when cooled, and then returns to its original shape when reheated; (5) improved cell formation in foams (stronger cell walls limit the cell-size distribution and prevent cells from blowing open); (6) improved solvent resistance (though some solvents cause the crosslinked material to swell); (7) better electrical resistance and dielectric properties; and/or any combination thereof.

In one exemplary embodiment, a surgical kit can contain a plurality of superior final implant and a plurality of inferior final implants. The said final inferior implant components may comprise three (3) lengths (Short, Medium, and Long), wherein each length of the inferior final implant component can be paired with 5 height sizes of the superior final implant (e.g., sizes 11, 12, 13, 14 and 15, wherein the height of each component differs by 1 mm). The superior final implant component can comprise a metal/polymer assembly that is machined from CoCr alloy, with a plasma sprayed Ti coating applied to at least a portion of at least one surface or a top surface thereto. The ASP is then molded to the CoCr portion, and the superior assembly is exposed to an initial or first dose of gamma radiation, which crosslinks and/or swells the preformed or unfinished implant and/or ASP to a desired degree. The preformed or unfinished implant and/or ASP is then machined to create at least one articulation or bearing surface and a plurality of bumpers or contact surfaces. In a final step, the superior and inferior final implant components can be inspected, cleaned, packaged, labeled, and gamma sterilized with a second dose of gamma radiation, which is desirably a sufficient level of gamma radiation to sterilize the component and/or obtain a final level of crosslinking, but which minimizes disruption of the machined surfaces and/or unacceptable swelling of the preformed or formed/final ASP.

In another exemplary embodiment, a surgical kit can include a 4.5 mm diameter (or other suitable dimeter) screw in the package that increases in length from 35 mm, to 40 mm, and 45 mm for the corresponding 3 lengths of inferior final Implant. The inferior final implant can be machined from bulk CoCr alloy, with a Ti coating applied after machining. In various embodiments, the surgical kit can further include a retainer clip that is manufactured (e.g., wire EDM) from a metal, the metal comprises CoCr. In a final step, the Inferior final Implant is cleaned, packaged with a screw or without a screw, labeled, and gamma sterilized.

One surprising advantage from incorporating a Vitamin E infused crosslinked UHMWPE material into the base element of a spinal element device arises from the inherent flexibility of this highly durable polymer material. Unlike metal-on-metal articulating implants commonly utilized in spinal implants, the metal on poly articulation facilitated by the disclosed implant designs desirably allow some or all of the polymeric material to “flex” or otherwise deform to some degree during periods of high or unusual loading, which greatly reduces any opportunity for the implant to lock or “freeze” during implant motion, as well as significantly reducing forces acting between the implant components and the spinal anatomy to which they are attached (which can undesirably loosen and/or separate the implant components from the underlying anatomy). Moreover, where a plurality of articulating implants may be operating concurrently, such as where a pair of articulating spinal implants may have been inserted into a single spinal level, the polymeric materials can desirably accommodate any mis- or mal-alignment between the two (or more) implants and/or their respective motion arcs, greatly reducing and/or eliminating any potential binding of or between the implants and/or significantly reducing implant wear over an extended period of time.

In the disclosed embodiments, the manufacturing of a final implant with a antioxidant stabilized polymer can desirably provide the final implant with an improved ability to accommodate or “tolerate” varying degrees of potential malalignment between the medial and lateral implant assemblies and accommodate the varying compressive forces while in-vivo use. In these embodiments, a superior final implant and an inferior final implant assemblies are deployed between a pair of vertebrae to establish a mobile system. Desirably, the implant assemblies can work in unison to allow movement of the vertebrae coupled thereto.

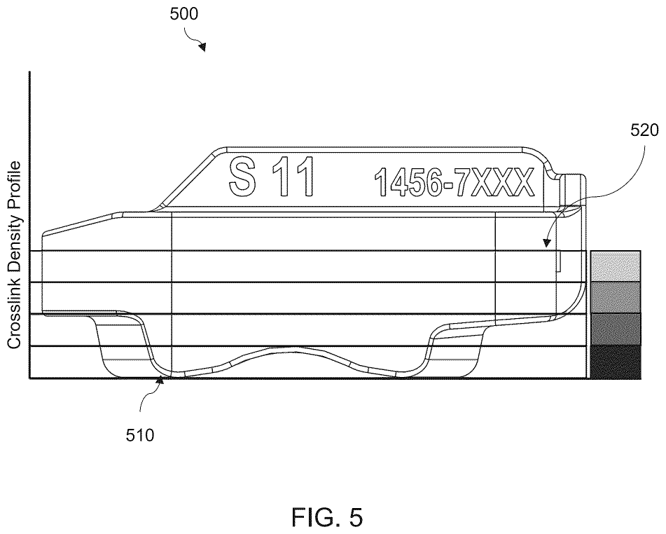

In one exemplary embodiment, shown in FIG. 5, a unique “step” or decreased crosslinking profile of the final or formed ASP of a superior final implant 500 (which interacts with an inferior final implant 600—see FIGS. 6A and 6B) can allow relevant portions of the final or formed ASP to flex and/or deform under varying loading conditions (e.g., compression, flexion, extension, torsion and/or lateral bending). The crosslink density can be highest at or proximate an inferior facing surface 510 (e.g., a final or formed ASP/bone interface) of the final or formed ASP of the final superior implant, and lowest at or proximate to an upper portion 520 of the superior base element/final or formed interface.

Furthermore, the final or formed ASP also behaves similar to a “shock absorber” under varying loading conditions, thereby minimizing stresses between the implant/bone interfaces, reducing any potential for “binding” of the paired implants, reduce wear debris and permitting the implant assemblies to be placed at a wide variety of relative positions and/or orientations within a targeted anatomical space. This arrangement can provide a better performing joint which experiences less wear and/or generates less wear debris than many other implant designs, as the concave UHMWPE “cup” can easily conform to or otherwise accommodate the harder ball or other convex surface, providing better wear performance and reducing the potential for stress concentrations and/or point loading of the joint. Similarly, the UHMWPE bumpers or stops can provide a cushioning force when the implant reaches a desired end of motion, rather than an abrupt stop which may be more prevalent with metal-on-metal contact surfaces. Accordingly, the superior and inferior final implant components of differing sizes and/or shapes to be utilized in combination within a single or multiple spinal level-giving greater flexibility to the surgeon to address unique patient anatomy and restore a desired sagittal balance to treated spinal levels.

### Results

Manufacturers recognize that different customers may have different criteria regarding the acceptable combination of oxidation resistance, wear resistance, and other important mechanical properties (such as fatigue resistance), and these criteria may differ for different types of implants for a given user or manufacturer. Therefore, the coordination of various method steps described herein may produce unexpected and/or desirable results for a given indications and/or surgical objective. In the present embodiments, the coordination of the method steps to create or form a cross-linked stabilized spinal implant was carefully balanced to desirably provide an optimal spinal implant with improved wear resistance in combination with other desired physical or chemical properties.

Table 1 below describes a combination of mechanical, chemical, and thermal characterization testing of the non-aged superior final implant that comprises vitamin E. The following tests were completed: (1) Tensile properties (yield stress, ultimate stress, percent elongation, ultimate tensile strength) (ASTM D638-14); (2) Compression properties (compressive modulus, Poisson's ratio) (ASTM D695); (3) IZOD impact energy (Annex A1 of ASTM F648 and ASTM D256); (4) Freeze fracture morphology; (5) Fatigue crack propagation (Paris regime exponent (m) and coefficient (C) and ΔK inception) (using ASTM E647-15e1 as a guide); (6) Density (ASTM D792); (7) Residual free radical concentration using electron spin resonance (in accordance with the 3rd edition of the UHMWPE Handbook); (8) Thermal properties using differential scanning calorimetry (onset and peak melting temperatures, heat of fusion, percent crystallinity) (ASTM 3418-12e1).

TABLE 1
Non-Aged Characterization Testing

| Material Properties | VE-UHMWPE (Non-Aged) |
|---|---|
| Tensile Yield Stress [MPa] | 22.3 ± 0.2 |
| Tensile Ultimate Stress [MPa] | 38.8 ± 3.9 |
| Tensile Percent Elongation [%] | 326.6 ± 22.1 |
| Compressive Modulus [MPa] | 1007 ± 38 |
| Poisson's Ratio | 0.41 ± 0.05 |
| Izod Impact Resistance [J/m] | 246.4 ± 19.4 |
| Izod Impact Strength [kJ/m²] | 87.0 ± 2.6 |
| Exponent (m) | 6.75 ± 0.75 |
| Coefficient (C) | 3.10 × 10⁻⁶ ± 9.08 × 10⁻⁷ |
| ΔK [MPa√m] | 1.3-1.5 |
| Density [kg/m³] | 936 ± 0.1 |
| Free Radical Concentration [spins/g] | 1.2 × 10¹⁶ ± 5.9 × 10¹³ |
| Onset Temperature of Melting [° C.] | 120.0 ± 0.2 |
| Melting Temperature [° C.] | 129.3 ± 0.1 |
| Heat of Fusion [J/g] | 145 ± 1 |
| Crystallinity [%] | 50 +/− 0.5 |

Table 2 below describes mechanical and chemical characterization of the superior final implant that comprises vitamin E after accelerated aging per ASTM F2003. The superior final implant was subjected to the process disclosed herein. The following tests were completed: Small punch testing properties (yield strength, ultimate elongation, ultimate strength, energy to failure) (ASTM F2977); (2) Oxidation Index (ASTM F2102); (3) TVI (ASTM F2381); (4) and Vitamin E Index (ASTM F2759) using Fourier transform infrared spectroscopy (FTIR); (5) Swell ratio and crosslink density (ASTM F2214-02).

TABLE 2
Aged Characterization Testing

| Material Properties | VE-UHMWPE (Aged) |
|---|---|
| Peak Load [N] | 69.2 +/− 3.9 |
| Ultimate Load [N] | 78.6 +/− 3.5 |
| Ultimate Displacement [mm] | 4.4 +/− 0.2 |
| Energy to Failure [mJ] | 244 +/− 15 |
| Maximum Oxidation Index | 0.13 +/− 0.02 |
| Maximum Transvinlyene Index | 0.033 +/− 0.001 |
| Vitamin E Index | Below Detection |
| Swell Ratio | 3.59 +/− 0.09 |
| Crosslink Density [mol/dm³] | 0.149 +/− 0.007 |

Table 3 below describes wear testing of the complete spinal implant that comprises a superior final implant including vitamin E and the inferior final implant. Wear testing was completed at 1 MC, 5 MC and 10 MC. The following wear tests were completed using the Standard Guide for Functional, Kinematic, and Wear Assessment Total Disc Prosthesis. Using the recommendations in this standard, an in vitro test was designed and used to evaluate wear performance.

TABLE 3
Wear Testing

| Cycles | Superior Final Implant (mg/MC) |
|---|---|
| 1.0 MC Wear Rate | 1.7 +/− 1.1 |
| 5.0 MC Wear Rate | 1.1 +/− 0.6 |
| 10.0 MC Wear Rate | 1.2 +/− 0.5 |

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

It should be understood that method(s) for manufacturing the various material mixture formulations and implants, including UHMWPE blends and/or surgical devices and related components and/or implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

I/We claim:

1. A spinal implant assembly comprising:
a superior component, the superior component comprises an upper base and an upper articulation component, the superior articulation component comprises a polymer material and a stabilizing component, the superior articulation component further comprises a concave surface; and
an inferior component, the inferior component comprises a base, an inferior articulation component, and a bridge, the inferior articulation component comprises a convex surface, the bridge extends from the base towards the posterior direction,
wherein the concave surface of the superior articulation component of the superior component contacts the convex surface of the inferior articulation component of the inferior component to create an articulation joint.

2. The spinal implant assembly of claim 1, wherein the polymer material comprises ultra high weight molecular polyethylene (UHWMPE).

3. The spinal implant of claim 1, wherein the polymer material comprises a cross-linked ultra-high weight molecular polyethylene (UHWMPE).

4. The spinal implant assembly of claim 1, wherein the stabilizing component comprises Vitamin E.

5. The spinal implant assembly of claim 1, wherein the polymer material comprises a cross-linked ultra-high weight molecular polyethylene (UHWMPE) and the stabilizing component comprises Vitamin E.

6. The spinal implant assembly of claim 3, wherein the Vitamin E comprises at least 900 parts per million of the vitamin E.

7. The spinal implant assembly of claim 3, wherein the Vitamin E comprises at least 0.02 percent by weight of the vitamin E.

8. The spinal implant of assembly of claim 3, wherein the Vitamin E comprises a Vitamin E uniformity depth within the superior articulation component, the uniformity depth extending from an exposed surface of upper articulation component to a depth of at least 3 millimeters.

9. The spinal implant of claim 5, wherein the cross-linked UHWMPE comprises a non-uniform cross-link density along a thickness or height of the crosslinked UHWMPE.

10. The spinal implant of claim 5, wherein the cross-linked UHWMPE comprises a crosslink profile, the crosslink profile changes from highest at or proximate to the inferior facing surface towards the superior direction.

11. A method of forming a stabilized spinal implant comprising:
creating an antioxidant stabilized polymer (ASP);
completing a first processing of the ASP to create a preformed implant;
completing a first cross-linking of the preformed implant using a selected first technique with a first dose and a first controlled environment;
completing a second processing of the preformed implant to create a final implant;
packaging the final implant in a packaging container within a packaging controlled environment; and
completing a second cross-linking and sterilization of the final implant with a selected second technique with a second dose and a second controlled environment.

12. The method of claim 11, wherein the step of creating an ASP comprises a blending process.

13. The method of claim 12, wherein the ASP comprises an antioxidant and a polymer.

14. The method of claim 13, wherein the antioxidant comprises α-tocopherol and the polymer comprises ultrahigh molecular weight polyethylene (UHWMPE).

15. The method of claim 14, wherein antioxidant comprises a concentration, the concentration includes at least 0.1% or greater.

16. The method of claim 11, wherein the first processing comprises direct compression molding.

17. The method of claim 11, wherein the first crosslinking technique comprises gamma beam.

18. The method of claim 11, wherein the second crosslinking technique comprises gamma beam.

19. The method of claim 10, wherein the second crosslinking technique is the same as the first crosslinking technique.

20. The method of claim 11, wherein the first dose of the first crosslinking technique is different than the second dose of the second crosslinking technique.
